SEQUENCE LISTING (3 sequences)

SEQ ID NO: 1 - DNA, 235 bp, Artificial Sequence
  synthetic oligonucleotide
atggctggcagatcatctcttcctcctctggggctacccctacctcaaaggaacagagtggcagcagtaccaatggcagcaatggcagtgagtcttccaagaatcgcacagtctctggtgggcagtatgttgtggctgccgctcccaacttacagaaccagcaagttctgacaggactacctggagtgatgcctaatattcagtatcaagtaatcccacagttccagaccgttga

SEQ ID NO: 2 - DNA, 22 bp, Artificial Sequence
  synthetic oligonucleotide
attcgatcggggcggggcgagc

SEQ ID NO: 3 - DNA, 22 bp, Artificial Sequence
  synthetic oligonucleotide
attcgatcgtagcgatgcgagc

(19) United States
(12) Patent Application Publication
(10) Pub. No.: US 2002/0137715 A1
(43) Pub. Date: Sep. 26, 2002

(54) Blocking Sp1 transcription factor broadly inhibits extracellular matrix gene expression in vitro and in vivo: implications for the treatment of tissue fibrosis

(76) Inventor: Alain Mauviel, Paris (FR)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/026,341

(22) Filed: Dec. 21, 2001

Related U.S. Application Data
(60) Provisional application No. 60/259,585, filed on Jan. 3, 2001.

(51) Int. Cl. A61K 48/00

(52) U.S. Cl. 514/44

(57) ABSTRACT

Sp1 is a transcription factor that is involved in the basal expression of ECM genes and is therefore important in fibrotic processes. The present invention relates to methods wherein transcriptional repression of the Sp1 gene by antisense Sp1 is used to reduce the expression of several ECM genes, without significant alteration in cell growth. The present invention further relates to decoy Sp1 binding oligonucleotides that interfere with Sp1 binding to its target DNA, thereby inhibiting ECM gene promoter activity both in vitro and in vivo. Targeting Sp1 will therefore allow for the efficient inhibition of ECM gene expression, and thereby allow for an alternative, non-toxic, therapeutic approach in the treatment of fibrotic disorders.

### CROSS REFERENCE TO RELATED APPLICATIONS

This application claims priority under 35 U.S.C. §119 based upon U.S. Provisional Patent Application No. 60/259,585 filed Jan. 3, 2001.

### GOVERNMENT RIGHTS TO THE INVENTION

This invention was made with government support under grants R29-AR43751, RO1-AR41439 and PO1-AR38923 awarded by the National Institute of Health. The government has certain rights in the invention.

### FIELD OF THE INVENTION

The present invention relates to the fields of molecular biology and genetics, and to a method of treating fibrotic conditions or disorders and, more particularly, to the inhibition of Sp1 activation of extracellular matrix protein gene expression.

### BACKGROUND OF THE INVENTION

Sp1 was one of the first eukaryotic transcription factors to be identified and cloned as a factor binding the SV40 early promoter. (Dynan and Tjian, Cell 35: 79-87, 1983). It is the founding member of a family of proteins with highly homologous zinc-finger domains in the C-terminal region that bind GC or GT boxes, while the glutamine rich domains in the N-terminus are essential for transcriptional activation (Kadonaga et al, Cell, 51: 1079-1090, 1987; Imataka et al, EMBO J. 11;3663-3671. 1992; Kingsley and Winoto, Mol Cell Biol 12: 4251-4261, 1992). Sp1 activates transcription by association with one of the co-activators associated with the TATA-binding protein (TBP) in the TFIID complex. In the case of TATA-less promoters, Sp1 is able to recruit the TATA-binding protein and associated TFIID, thereby positioning the initiation complex to the correct start site even in the absence of a TATA box. (Pugh and Tjian, Genes Dev 5: 1935-1945, 1991; Wu et al, J Biol Chem 269: 28450-28459, 1994). Other suggested roles for Sp1 in nuclear processes include remodeling of chromatin structures (Jongstra et al, Nature 307: 708-714, 1984) and maintenance of methylation-free CpG islands. (Brandeis et al, Nature, 371: 435-438,1994; MacLeod et al, Genes Dev 8: 2282-2292,1994). Therefore, Sp1 is fundamental for the establishment of transcriptional competence, in addition to its role as a transcription factor.

In a majority of promoters containing Sp1 binding sites, Sp1 provides a basal level of transcription. (reviewed in Cook et al, Ann N Y Acad Sci 880 :94-102, 1999). It plays an important role in the expression of numerous elements of the cell-cycle machinery, such as cyclins, Rb-like proteins, and E2F. (Shao and Robbins, Oncogene 10: 221-228, 1995; Karlseder et al, Mol Cell Biol 16: 1659-1667, 1996; Lin et al, Mol Cell Biol 16: 1668-1675, 1996). Targeted disruption of the mouse Sp1 gene has shown that Sp1 is critical for normal embryogenesis. Sp1^{-/-} embryos are severely retarded in their development and display a marked heterogeneity in their phenotype. (Marin et al, Cell 89: 619-628, 1997). Interestingly, inactivation of the Sp1 gene is compatible with a certain degree of cell growth and differentiation, and the expression of various putative target genes, including that of certain cell cycle-related genes, was not altered in Sp1^{-/-} embryos. Also, CpG islands remained methylation free and active chromatin was formed at the globin loci. This may occur possibly because other members of the Sp1 family partially compensate for the absence of Sp1, thereby ameliorating the Sp1 knockout. (Marin et al, Cell, 89: 619-628, 1997).

Type I collagen and extracellular matrix (ECM) accumulation is one of the hallmarks of fibrotic conditions. For example, affected skin areas from patients with systemic sclerosis exhibit abnormal accumulation of various ECM components. predominantly types I and III collagens, but also types V and VII, as well as various proteoglycans. (Ishikawa and Horiuchi, Dermatologica 150: 334-345, 1975; Uitto, J Invest Dermatol 72: 1-10, 1979; Perlish et al, J Invest Dermatol 90: 48-54,1988). The accumulation of ECM proteins is accompanied by elevated mRNA steady-state levels of fibrillar collagens. (Kähäri et al, Biochem Biophys Acta 781: 183-186, 1984. Ohta and Uitto, Arthritis Rheum 30: 404-411, 1987; Scharfetter et al, Eur J Clin Invest 18: 9-17, 1988).

Several studies have emphasized the importance of Sp1 and cognate cis-elements in the expression of the genes encoding the α1 and α2 chains of type I collagen in several species. (Pogulis and Freytag, J Biol Chem 268: 2493-1499. 1993: Tamaki et al, J Biol Chem 270: 4299-4304, 1995; Ihn et al, J Biol Chem 271: 26717-26723, 1996; Chung et al, J Biol Chem 271: 3272-3278, 1996; Miao et al, Arch Biochem Biophys 341: 140-152, 1997; Artlett et al, Matrix Biol 17: 425-434, 1998). Also, there are some indications that Sp1 transcription factor may be involved in the excessive expression, and resulting deposition, of type I collagen in fibrotic conditions. For example, increased Sp1 binding activity to the COL1A1 promoter has been observed in fibroblasts derived from affected skin from patients with systemic sclerosis. (Hitraya et al, Arthritis Rheum 41: 2048-2058, 1998). Similarly, fibrogenic stimuli induce Sp1 DNA binding activity in Ito cells, a phenomenon which may contribute to the development of liver fibrosis. (Rippe et al, Hepatology 22: 241-251, 1995). Also, Sp1 DNA binding activity is increased (from 30 minutes to 12 weeks post-irradiation) in a model of gamma-irradiation-induced lung fibrosis in the rat. (Haase et al, Int J Radiat Biol 76: 487-492, 2000).

The present invention relates to targeting Sp1 to simultaneously block the transcription of several ECM genes. The invention disclosed herein presents novel therapeutic approaches toward treatment of fibrotic disorders. A combination of cDNA array/gene promoter analysis/decoy oligonucleotide technologies is used to show that interfering with Sp1 expression or function leads to simultaneous transcriptional inhibition of the expression of several collagen, proteoglycan, and other ECM genes in fibroblasts. The present invention provides a proof of concept for a gene therapy-based approach against tissue fibrosis, using the Sp1 transcription factor as a target.

### ABBREVIATIONS

“CAT” is chloramphenicol acetyl transferase

“ECM” is extracellular matrix

“SL2 cells” is Drosophila Schneider L2 cells

“TGF-β” is transforming growth factor-β

### SUMMARY OF THE INVENTION

It is an object of the present invention to present a method of treating a fibrotic condition wherein the transcription of an ECM gene is inhibited in a mammal. A therapeutically effective amount of antisense Sp1 is administered to the mammal, the antisense Sp1 binds to an Sp1 transcript, reduces Sp1 expression and inhibits transcription of the ECM gene with the subsequent reduction in the accumulation of the corresponding ECM protein. In one embodiment of the method of treating the fibrotic condition, the antisense Sp1 has the nucleic acid sequence of SEQ. ID. NO: 1.

It is another object of the invention to present a recombinant expression vector that contains a XhoI/HindIII DNA fragment of an Sp1 gene (SEQ. ID. NO: 1) In an antisense orientation cloned upstream of a Rous Sarcoma Virus (RSV) promoter.

It is an object of the present invention to also present to a method of treating a fibrotic condition wherein the gene expression of an ECM gene is inhibited in a mammal. A therapeutically effective amount of a decoy Sp1 oligonucleotide is administered to the mammal, the Sp1 transcription factor binds to the decoy Sp1 oligonucleotide, thereby interfering with Sp1 binding to its target sequence(s). In one embodiment the interference of the binding of the Sp1 transcription factor to its target sequence(s) by the decoy oligonucleotide causes a decrease in the promoter activity of the ECM gene, inhibiting gene expression, and reducing the accumulation of the corresponding ECM protein. In a further embodiment of the method of treating a fibrotic condition the decoy Sp1 oligonucleotide has the nucleic acid sequence of SEQ. ID. NO: 2.

In another embodiment the interference of the binding of the Sp1 transcription factor to its target sequence(s) by the decoy oligonucleotide causes a decrease in the promoter activity of a TGF-β gene, inhibiting gene expression of the TGF-β gene, and blocking the fibrogenic properties of TGF-β. In one embodiment the decoy Sp1 oligonucleotide has the nucleic acid sequence of SEQ. ID. NO: 2.

### DESCRIPTION OF THE DRAWINGS

FIG. 1. Stable expression of an antisense Sp1 vector efficiently reduces Sp1 DNA binding activity in NIH-3T3 fibroblasts. A, Nuclear extracts are prepared from clones of NIH 3T3 stably transfected with either empty pRSV (clone 0, lane 1) of pRSV/ASSp1 (clones A-E, lanes 2-6). Sp1 DNA binding activity is determined by EMSA, using a radiolabeled consensus Sp1 oligonucleotide. Note that protein/DNA complex formation is dramatically reduced in lane 3 (clone B). B, Supershift assays using nuclear extracts from either clone 0 (lanes 1, 2) or clone B (lanes 3, 4) are carried out as described (infra).

FIG. 2. Select results from differential hybridizations of Atlas™ human cell interaction cDNA expression arrays: effect of antisense Sp1 expression. Differential hybridization of cDNA expression arrays is performed (infra). A, NIH 3T3 fibroblast genes inhibited at least 2-fold by stably transfected pRSV/ASSp1. B, genes whose expression is not altered by the antisense.

FIG. 3. Inhibition of COL1A2 promoter activity by antisense Sp1 correlates with the number of Sp1 binding sites. Fibroblasts in late logarithmic growth phase are transfected with 5 μg of various 5′ deletion constructs of the COL1A2/CAT promoter and 10 μg of either empty pRSV (open bars) or pRSV/ASSp1 (solid bars). Forty hours later, cell extracts are prepared and assayed for CAT activity. The relative CAT activity (mean±SD) of three independent experiments performed in duplicate is shown.

FIG. 4. Contribution of Sp1 sites to various ECM gene promoter transactivaition. Sp1-deficient Drosophila SL2 cells are co-transfected with different 5′ end deletion constructs of various ECM gene promoters linked to the CAT gene, together with either empty pPac0 (open bars) or pPacSp1 Sp1 expression vector (solid bars). 40 h after transfection, cell extracts are assayed for CAT activity. The relative promoter activities (mean±SD) from three independent experiments performed in duplicate is shown in the form of a bar graph. The position of the 5′ end of each construct is indicated below each histogram bar. A, COL1A2, B, COL3A1, C, decorin, D, TIMP-1.

FIG. 5. Decoy Sp1 oligonucleotides inhibit COL1A2 promoter activity in vitro and in vivo. A, Confluent human dermal fibroblast cultures are transfected with 5 μg of −500COL1A2/CAT reporter gene construct. After glycerol shock, increasing amounts (2, 5, 10 μg) of Sp1 (open bars) or mSp1 (hatched bars) oligonucleotides are added to the culture medium. Forty hours later, CAT activity is determined and compared to that of cultures without oligonucleotides (closed bar). Relative promoter activity (mean±SD) of three separate experiments performed in duplicate is presented. B, Four transgenic mice carrying 17 kb of mouse COL1A2 promoter linked to the luciferase gene in their genome are injected subcutaneously at distant sites (approx. 2 cm apart) with 15 μg of either Sp1 (open bar) or mSp1 (hatched bar) oligonucleotides in 25 μl of H₂O. After a 30-hr incubation, animals are sacrified and luciferase activity is determined in the skin surrounding the injection sites using identical amounts of protein extracts (40 μg). A non-injected skin area is used as control (solid bar).

### DESCRIPTION OF THE INVENTION

Materials and Methods

Cell cultures. Human dermal fibroblasts and NIH 3T3 fibroblast cultures are grown in Dulbecco's modified Eagle's medium supplemented with 10% heat-inactivated fetal calf serum, 2 mM glutamine, and antibiotics (100 units/ml penicillin, 50 μg/ml streptomycin-G, and 0.25 μg/ml Fungizone™). Sp1-deficient cells (Drosophila melanogaster Schneider L2 cells) are grown in Schneider's Drosophila medium (Life Technologies, Inc., Paisley, Scotland).

Plasmid constructs. Several ECM gene promoter/reporter gene constructs are used to examine the role played by Sp1 in their transcriptional activity. The human COL1A2, COL7A1, and decorin promoter constructs have been described previously. (Mauviel et al, J Biol Chem 270: 11692-11700, 1995; Chung et al, J Biol Chem 271: 3272-3278, 1996; Vindevoghel et al, J Biol Chem 272: 10196-10204, 1997; Higashi et al, Matrix Biol 16: 447-456, 1998; Kouba et al, J Immunol 162: 4226-4234, 1999). Human COL1A1 (Li et al, Gene 164, 229-1234, 1995), mouse COL3A1 (Mudryj and de Crombrugghe, 1988), human COL5A2 (Truter et al, J Biol Chem 267: 25389-25395, 1992), and human TIMP-1 (Clark et al, Biochem J 324: 611-617. 1997) promoter constructs are kind gifts from Drs. S. Jimenez (Thomas Jefferson University, Philadelphia, Pa.), B. de Crombrugghe (M. D. Anderson Cancer Center. Houston, Tex.), F. Ramirez (Mount Sinai Medical Center, New York, N.Y.), and D. R. Edwards (University of Calgary, Calgary, Alberta, Canada), respectively.

pPacSp1, an expression vector for Sp1 driven by the actin promoter (Courey and Tjian, Cell 55: 887-898, 1988), a kind gift from Dr. R. Tjian (University of California, Berkeley, Calif.), is used to express Sp1 in Drosophila SL2 cells. Empty pPac0 is used as a control.

pRSV/ASSp1 is generated by cloning a PCR-generated XhoI/HindIII DNA fragment spanning the region +47 to +281 of the human Sp1 gene (Genbank # J03133) in an antisense orientation upstream of the Rous Sarcoma Virus (RSV) promoter, into pRSV expression vector. The integrity of the construct is verified by automated sequencing.

Generation of stably transfected fibroblasts. NIH 3T3 fibroblast cultures are transfected with pRSV/ASSp1 together with pCMVneo, a plasmid that expresses the neomycin-resistance gene under the control of the cytomegalovirus (CMV) promoter. at a 10/1 ratio using the calcium phosphate/DNA co-precipitation method. Four days after transfection, Geneticin™ (Sigma Chemical Co., St-Louis, Mo.), 0,8 mg/ml, is added to the culture medium to allow selection of transfected cells. After 15 to 20 days, several clones are isolated and propagated in maintenance medium containing 0,4 mg/ml of Geneticin™.

Electrophoresis mobility shift assays (EMSA). A 22-mer consensus Sp1 oligonucleotide 5′-ATTCGATCGGGGCGGGGCGAGC-3′ was used as a probe to determine Sp1/DNA interactions. For supershift experiments, nuclear extracts (5 μg) are incubated for 2 h on ice with an anti-Sp1 antibody (Santa Cruz Biotechnologies, Santa Cruz, Calif.) prior to the binding reactions. Binding mixtures are separated electrophoretically on native 4% acrylamide gels, as described previously (Vindevoghel et al, 1997).

Transient cell transfection and CAT assays. Transient cell transfections are performed with the calcium phosphate/DNA co-precipitation procedure. CAT activity is measured using [¹⁴C]chloramphenicol as substrate (Gorman et al, Mol Cell Biol 2: 1044-1051, 1982) followed by thin layer chromatography and quantitation with a phosphorimager.

Differential hybridization of Atlas™ human cDNA expression arrays. Total RNA is obtained using an RNeasy kit (Qiagen GmbH, Hilden Germany). DNAase I treatment is performed on the RNA samples directly on the spin columns to avoid genomic DNA contamination. Radioactive cDNA synthesis is carried out as described in the Atlas™ cDNA expression arrays user manual (Clontech, San Diego, Calif.). Equal amounts of radiolabeled cDNAs (10⁷ cpm) from clones transfected with either pRSV/ASSp1 or empty pRSV are hybridized in parallel to two Atlas™ cDNA expression arrays for 18 hours at 68° C. The mouse-specific Atlas™ expression arrays (Clontech #7741-1) are washed four times in 2×SSC and 1% SDS for 30 minutes at 68° C. and twice in 0,1×SSC and 0,5% SDS at 68° C., according to the manufacturer's protocole. Atlas™ human cell interaction arrays (Clontech #7746-1) hybridized with mouse RNA-derived radiolabeled cDNAs are washed in the same solutions as above, but at a temperature of 52° C., which is determined to provide the best signal/background ratio. Membranes are then exposed to Kodak phosphor screens for three days. Hybridization signals are quantified with a Storm 840 phosphorimager using the ImageQuant software (Amersham Pharmacia Biotech), and normalized against glyceraldehyde-3-phosphate dehydrogenase (GAPDH) mRNA levels in the same samples. Significance of inhibition of gene expression is set arbitrarilly to 2-fold.

Decoy oligonucleotides. In some in vitro experiments decoy double-stranded oligonucleotides are used in an attempt to interfere with Sp1 binding to its cognate cis-elements within the COL1A2 promoter. Specifically, a 22-mer consensus Sp1 oligonucleotide, 5′-ATTCGATCGGGGCGGGGCGAGC-3′, is added to the culture medium of dermal fibroblasts transfected with −3500COL1A2/CAT. A mutant oligonucleotide, mSp1, 5′-ATTCGATCGTAGCGATGCGAGC-3′, is used as a control. 24 h later, CAT activity, representing promoter activity, is determined.

Treatment of transgenic mice. To test the effects of decoy Sp1 oligonucleotides on COL1A2 promoter activity in vivo, 15 μg of either Sp1 or mSp1 oligonucleotides in 25 μl of H₂O, are injected intradermally into 15-day old transgenic mice harbouring the luciferase gene under the control of 17 kb of the mouse COL1A2 promoter. (Bou-Gharios et al, J Cell Bio 134: 1333-1344, 1996). After 24 hours, the animals are sacrified and a ˜1 cm² area of the skin covering the site of injections is removed and homogenized with a tissue grinder into luciferase reporter lysis buffer (Promega Corp., Madison, Wis.). Luciferase activity using identical amounts of extracted proteins is determined with a commercial assay kit (Promega).

### Results

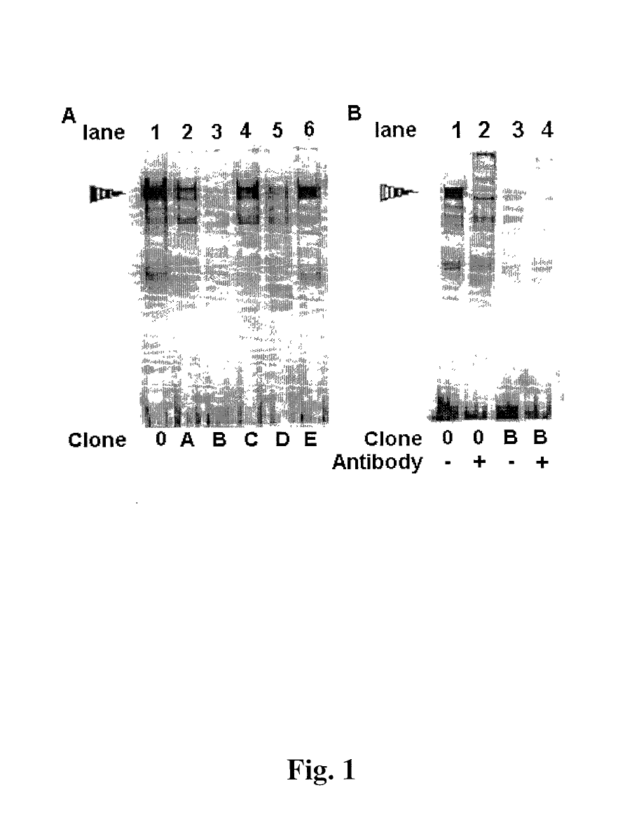

Stably transfected pRSV/ASSp1 efficiently reduces Sp1 expression in NIH 3T3 fibroblasts. Several clones transfected with the pRSV/ASSp1 antisense Sp1 expression vector are isolated and tested by EMSA for their Sp1 DNA binding activity. Although the amounts of DNA/protein complexes (arrow) in the nuclear extracts that are prepared from confluent plates from the different clones (FIG. 1A, A-E, lanes 2-6) varied significantly in their binding activity (FIG. 1A), they are always lower than the binding activity observed in a control clone (0) transfected with empty pRSV (FIG. 1A, lane 1). Phosphorimager quantitation of the radioactivity present in the various complexes reveals that clone B contains the least DNA binding activity (about 2% of the radioactivity present in complexes formed in the control nuclear extracts). An anti-Sp1 antibody efficiently supershifted the complex observed in the control extracts (FIG. 1B, lanes 1 and 2), as well as the residual Sp1/DNA complexes observed in clone B (FIG. 1B, lanes 3 and 4), attesting for their identity as Sp1/DNA complexes, as expected from previous work. (Vindevoghel et al, J Biol Chem 272: 10196-10204, 1997).

Despite little Sp1 transcription factor, clone B had growth capacities not discemable from that of clones transfected with empty pRSV or that of parental NIH 3T3 fibroblast cultures.

Effects of antisense Sp1 on fibroblast gene expression profiles as measured by cDNA microarray analysis. The technique of differential hybridization of Atlas™ cDNA expression arrays is used to identify differences in the expression pattern of 853 known genes between empty pRSV-transfected clone 0 and pRSV/ASSp1-transfected clone B. First, cDNA arrays containing probe sets for 588 known mouse genes are used. Among these genes, 266 of them do not show significant hybridization signal in either pRSV- or pRSV/ASSp1-transfected clones. Sixty five cellular genes are inhibited by more than 50% by pRSV/ASSp1 (Table I). This inhibitory effect is specific, as the expression of 257 genes is not, or only slightly modified. None of them are upregulated. Among the inhibited genes are several whose products are involved in the cell cycle (cyclins D1, D2, D3, cyclin-dependent kinase 5, for example). On the other hand, several other cell cycle genes are not affected (cyclin A, cyclin B1, cyclin-dependent kinase 4). This selective effect of pRSV/ASSp1 on cell-cycle-related genes may explain why the transfected clones exhibit a normal growth behavior.

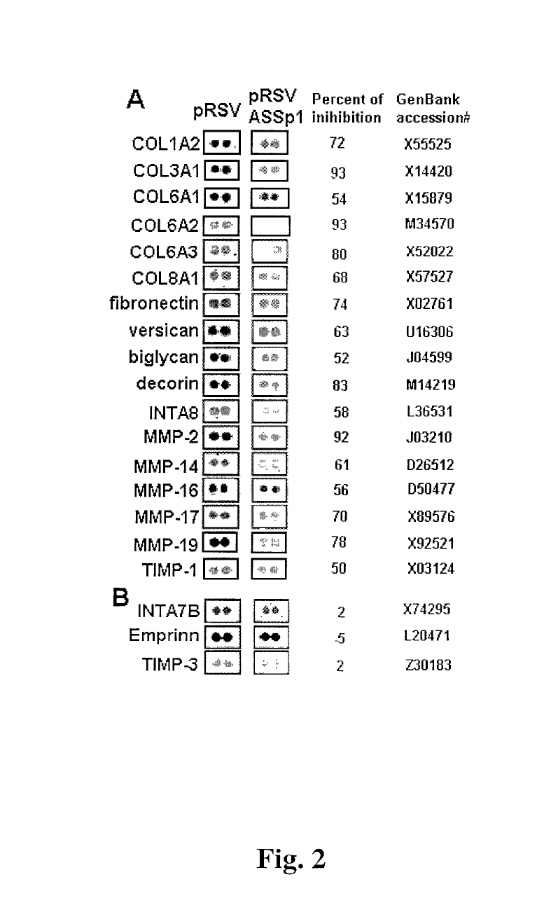

Next, to determine the effect of Sp1 gene targeting on ECM gene expression, cDNA arrays containing probe sets for 265 known human ECM genes are used. Signal/background ratios are optimized by testing various washing conditions of the arrays after hybridizations (supra). Significant expression levels are noted for 111/265 genes. Among them, 43 are inhibited at least 2-fold in pRSV/ASSp1-transfected clone B (Table II). Select hybridization results are presented in FIG. 2A, illustrating, the simultaneous reduction of steady-state mRNA levels for several ECM genes collagens (COL1A2, COL3A1, COL5A2, COL6At, COL6A2, COL6A3, COL8A1, COL1A2), proteoglycans (decorin, biglycan, versican), matrix metalloproteinases (MMP-2, MMP-14, MMP-16, MMP-17 and MMP-19), fibronectin, integrin alpha 8. and TIMP-1. The remaining 68 genes, including those for TIMP-3, integrin alpha 7 and emmprin for example (FIG. 2B) show little or no alteration in their expression levels.

TABLE I
Genes inhibited at least 2-fold by pRSV/ASSp1 in stably
transfected NIH 3T3 fibroblasts, as measured using differential
hybridization of Atlas ™ mouse cDNA
arrays (Clontech #7741-1)

| | |
|---|---|
| Accession | |
| n^{o} | Definition |

ONCOGENES AND TUMOR
SUPRESSORS

| | |
|---|---|
| X58876 | p53-regulating protein |
| J04115 | c-Jun proto-oncogene |
| M13071 | A-Raf proto-oncogene |
| M64429 | B-Raf proto-oncogene |
| M84607 | platelet-derived growth |
| | factor alpha-receptor |
| U28495 | Lfc proto-oncogene |
| U15784 | Shc transforming |
| | adaptor protein |
| D17584 | beta-protachykinin a |
| X67735 | Mas proto-oncogene |

CELL CYCLE
REGULATORS

| | |
|---|---|
| S78355 | cyclin D1 (G1/S-specific) |
| M83749 | cyclin D2 (G1/S-specific) |
| U43844 | cyclin D3 (G1/S-specific) |
| Z37110 | cyclin G (G2/M-specific) |
| D29678 | cyclin-dependent kinase 5 |
| U10440 | cdk inhibitor p27 |
| U09507 | cdk inhibitor p21/Waf1 |

STRESS RESPONSE
PROTEINS

| | |
|---|---|
| D49482 | Osp94 osmotic stress |
| | protein |
| J05186 | ERp72 endoplasmic |
| | reticulum stress protein |
| U41751 | etoposide induced p53 |
| | responsive (EI24) |
| | mRNA |
| D78645 | 78-kDa glucose |
| | regulated protein |
| U40930 | oxidative stress-induced |
| | protein mRNA |
| M10021 | dioxin-inducible |
| | cytochrome P450 |

HORMONE RECEPTORS

| | |
|---|---|
| X13358 | glucocorticoid receptor |
| | form A |
| M33324 | growth hormone |
| | receptor |
| J05149 | insulin receptor |

MODULATORS,
EFFECTORS AND
INTRACELLULAR
TRANSDUCERS

| | |
|---|---|
| M83336 | interleukin-6 receptor |
| | beta chain |
| S76657 | cAMP response element |
| | binding protein 1 |
| U06922 | Stat3 |
| M61177 | ERK1 |
| L33768 | Jak3 tyrosine-protein |
| | kinase |
| U03279 | phosphatidylinositol 3- |
| | kinase catalytic subunit |
| U20238 | GapIII; GTPase- |
| | activating protein |
| M21065 | interferon regulatory |
| | factor 1 |

APOPTOSIS-ASSOCIATED
PROTEINS

| | |
|---|---|
| M16506 | bcl2 |
| D30687 | glutathione S-transferase |
| | Pi 1 |
| U19463 | A20 zinc finger protein |
| U97076 | FLIP-L; apoptosis |
| | inhibitor |
| L28177 | gadd45 |
| U04710 | insulin-like growth factor |
| | receptor II (IGFR II) |
| X57796 | TNFR1 (55 kDa) |

DNA SYNTHESIS, REPAIR
AND RECOMBINATION
PROTEINS

| | |
|---|---|
| X66323 | ATP-dependent DNA |
| | helicase II 86-kDa |
| | subunit |
| X96618 | PA6 stromal protein |
| D64107 | MmLim15; RecA-like |
| | gene |

PROTEIN TURNOVER

| | |
|---|---|
| M14222 | cathepsin B |
| X53337 | cathepsin D |
| X06086 | cathepsin L |
| L28095 | interleukin-converting |
| | enzyme |

TRANSCRIPTION FACTORS
AND DNA-BINDING
PROTEINS

| | |
|---|---|
| D26046 | AT motif-binding factor |
| | ATBF1 |
| M58566 | butyrate response factor 1 |
| L21671 | epidermal growth factor |
| | receptor kinase substrate |
| | EPS8 |
| Z36885 | Ets-related protein Sap 1A |
| U20344 | Gut-specific Kruppel-like |
| | factor GKLF |
| J03770 | homeobox protein 4 2 |
| U62522 | Sp4 zinc finger |
| | transcription factor |
| U20532 | nuclear factor related to |
| | P45 NF-E2 |
| U09419 | retinoid X receptor |
| | interacting protein |
| X70298 | SRY-box containing gene 4 |
| X91753 | transcription factor SEF2 |

GROWTH FACTOR AND
CHEMOKINE RECEPTORS

| | |
|---|---|
| M28998 | fibroblast growth factor |
| | receptor basic |

GROWTH FACTORS,
CYTOKINES AND
CHEMOKINES

| | |
|---|---|
| X69619 | inhibin beta A subunit |
| U44725 | mast cell factor |
| L34169 | thrombopoietin |
| M13177 | TGF-β1 |

CELL SURFACE ANTIGENS
AND ADHESION
PROTEINSNEUROTRANSMITTER
RECEPTORS

| | |
|---|---|
| M27129 | CD44 antigen |
| X69902 | integrin alpha 6 |

TABLE II
ECM-related genes inhibited more than 2-fold by
pRSV/ASSp1 in stably transfected NIH 3T3 fibroblasts, as
measured using differential hybridization of Atlas ™
human cell interaction cDNA arrays
(Clontech #7746-1)

| | |
|---|---|
| Accession | |
| no. | Definition |

CELL-MATRIX
INTERACTIONS

| | |
|---|---|
| X55525 | collagen type I alpha-2 |
| X14420 | collagen type III pro- |
| | alpha-1 |
| X15879 | collagen type VI alpha-1 |
| M34570 | collagen type VI alpha-2 |
| X52022 | collagen type VI alpha-3 |
| X57527 | collagen type VIII alpha-1 |
| X02761 | fibronectin |
| U16306 | versican, isoforms |
| | V1,V2,V3 |
| J04599 | byglycan |
| M14219 | decorin |
| L36531 | integrin alpha8 |
| J02703 | integrin beta3 |
| J05633 | integrin beta5 |
| J03210 | MMP-2 |
| D26512 | MMP-14 |
| D50477 | MMP-16 |
| X89576 | MMP-17 |
| X92521 | MMP-19 |
| X03124 | TIMP-1 |

CELL-CELL
INTERACTIONS

| | |
|---|---|
| D12614 | lymphotoxin |
| | (TNF-beta) |
| L34060 | cadherin-8 |
| D13866 | alpha-catenin |
| X87838 | beta-catenin |
| Y11306 | beta-catenin/TCF-4 |
| L40636 | ephrin type-B |
| | receptor 1 precursor |
| L41939 | ephrin type-B |
| | receptor 2 precursor |
| U07695 | ephrin type-B |
| | receptor 4 precursor |
| M57730 | ephrin-A1 precursor |
| M73980 | Notch1 |
| U77493 | Notch2 |
| U46461 | dishevelled homolog |
| X97057 | WNT-10B |

CYTOPLASMIC REGULATOR
AND EFFECTORS

| | |
|---|---|
| Z18951 | caveolin-1 |
| U59752 | cytohesin-1 |
| L11353 | moesin-ezrin-radixin-like |
| | protein |
| Y07604 | nucleoside-diphosphate |
| | kinase |
| X17620 | nucleoside diphosphate |
| | kinase A |
| U11690 | putative rho/rac guanine |
| | nucleotide exchange |
| | factor |
| M29870 | RAS-related C3 |
| | butulinum toxin |
| | substrate 1 (P21-RAC1) |
| M64595 | RAS-related C3 |
| | butulinum toxin |
| | substrate 1 (P21-RAC2) |
| X69550 | rho GDP-dissociation |
| | inhibitor 1 |
| X06820 | rhoB |
| L25080 | proto-oncogene rhoA |
| | multidrug |
| | resistance protein |
| X94991 | zyxin + zyxin-2 |

Antisense Sp1 reduces the activity of various ECM promoter/CAT reporter gene constructs. The simultaneous reduction of steady-state mRNA levels for ECM genes by antisense Sp1 expression (supra) is observed using differential cDNA array hybridization. The present invention determines if this reduction of steady-state mRNA levels is a result of transcriptional inhibition at the level of their promoter regions. The 5′ regulatory regions of genes encoding fibrillar collagens (COL1A2, COL1A1, COL5A2 and COL3A1), TIMP-1, decorin, and type VII collagen (COL7A1) is analyzed for transciptional inhibition. Decorin is a small proteoglycan thought to play a role in type I collagen fibrillogenesis. (reviewed in Kuc and Scott, Connect Tissue Res 36: 287-296, 1997). COL7A1 is a basement membrane-associated collagen component of the anchoring fibrils, whose diffuse expression in the dermis has been observed in the affected skin from patients with systemic sclerosis and may contribute to its tightly bound and indurated appearance. (Rudnicka et al, J Clin Invest 93:1709-1715, 1994). As shown in Table III, all constructs tested have significantly reduced transciption when co-transfected with pRSV/ASSp1. These results indicate that the broad inhibition of mRNA steady-state levels resulting from inhibition of Sp1 expression occurs at the transcriptional level, consistent with the role of Sp1 as a transcription factor.

TABLE III
Antisense Sp1 expression vector inhibits the activity of
various ECM promoter/CAT reporter gene constructs

Promoter activity^{a}

| | |
|---|---|
| Promoter | Co-transfected vector |

| constructs | pRSV | pRSV/ASS | % of inhibition |
|---|---|---|---|
| −676COL1A1 | 32.9 ± 5.7 | 15.1 ± 1.1 | 54.1 ± 3.3 |
| −3500COL1A2 | 57.9 ± 5.3 | 15.3 ± 4.1 | 73.6 ± 7.1 |
| −400COL3A1 | 3.9 ± 0.1 | 0.6 ± 0.1 | 84.6 ± 12.0 |
| −2300COL5A2 | 4.3 ± 0.6 | 2.9 ± 0.4 | 32.5 ± 9.3 |
| −524COL7A1 | 6.1 ± 0.3 | 4.7 ± 0.2 | 22.9 ± 3.3 |
| −480decorin | 15.1 ± 0.8 | 3.5 ± 1.5 | 76.8 ± 9.9 |
| −850TIMP-1 | 74.6 ± 0.9 | 32.1 ± 2.5 | 56.9 ± 3.3 |

Confluent human dermal fibroblast cultures were transfected with 5 μg of various ECM promoter/CAT reporter gene constructs, together with 10 μg of either empty pRSV or pRSV/ASSp1. Fourty hours after transfection, cell extracts were assayed for CAT activity. Results presented as percent of acetylation are the mean±SD of three independent experiments performed with duplicate samples.

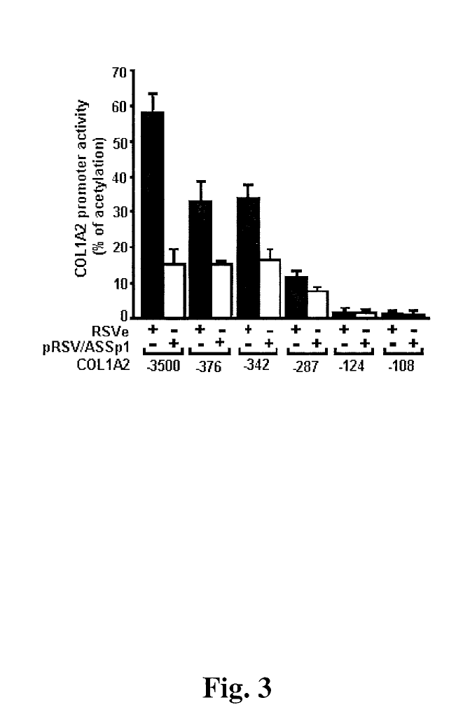

Modulation of Sp1 levels regulates promoter activity and this regulation is proportional to the number of Sp1 binding sites available. 5′-end deletion constructs of the human COL1A2 promoter are tested for their responsiveness to antisense Sp1 expression. As shown in FIG. 3, these constructs exhibit various degrees of basal activity, consistent with previous observations, and attributed to the presence of several functional Sp1 sites. (Inagaki et al, J Biol chem 269: 14828-14834, 1994; Chung et al, J Biol Chem 271: 3272-3278, 1996; Ihn et al, J Biol Chem 271: 26717-26723, 1996; Higashi et al, Matrix Biol 16: 447-456, 1998; Kouba et al, J Immunol 162: 4226-4234, 1999). In particular, integrity of the Sp1 sites located within the region −342/−271 of the human COL1A2 promoter is critical for maintenance of high promoter activity. (Inagaki et al, J Biol Chem 269: 14828-14834, 1994; Chung et al, J Biol Chem 271: 3272-3278, 1996; Ihn et al, J Biol Chem 271: 3272-3278. 1996), An 80% inhibition of the activity of the longest construct, −3500COL1A2/CAT, is observed. The extent of inhibition is reduced to about 50% for constructs −376 and −342, and drops to about 30% for construct −287. These data indicate that yet to be identified elements upstream of −376, between −376 and −3500, significantly contribute to Sp1-driven COL1A2 gene expression. Shorter constructs, −124 and −108COL1A2/CAT, have little activity and are not inhibited by the antisense Sp1 vector. A similar pattern of results is observed using deletion constructs of the promoters for decorin, COL3A1 and COL7A1, and TIMP-1. Together, these results indicate that the extent of inhibitory activity of pRSV/ASSp1 on a given promoter is directly proportional to the number of functional Sp1 binding sites.

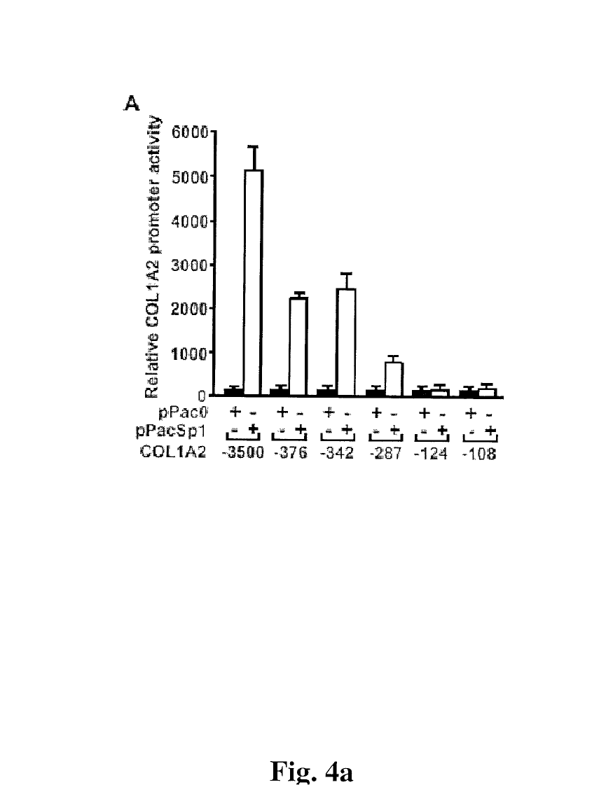

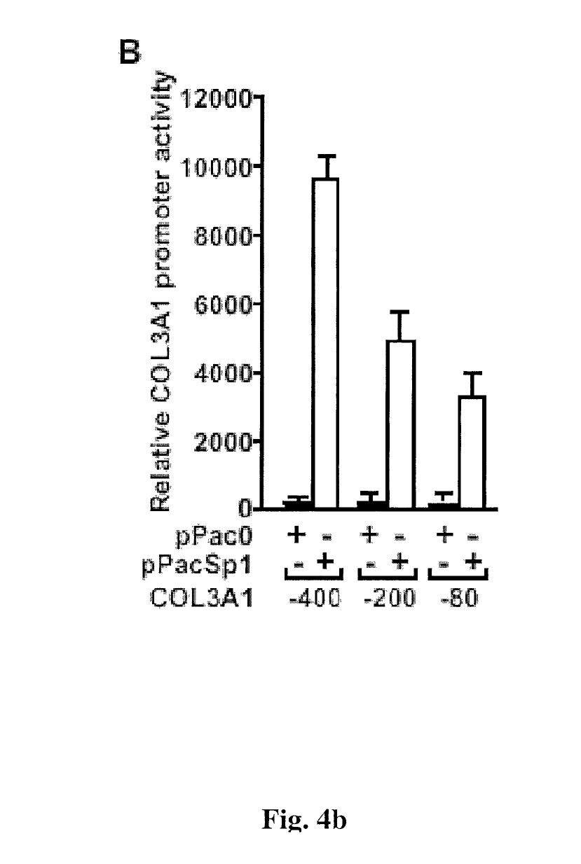

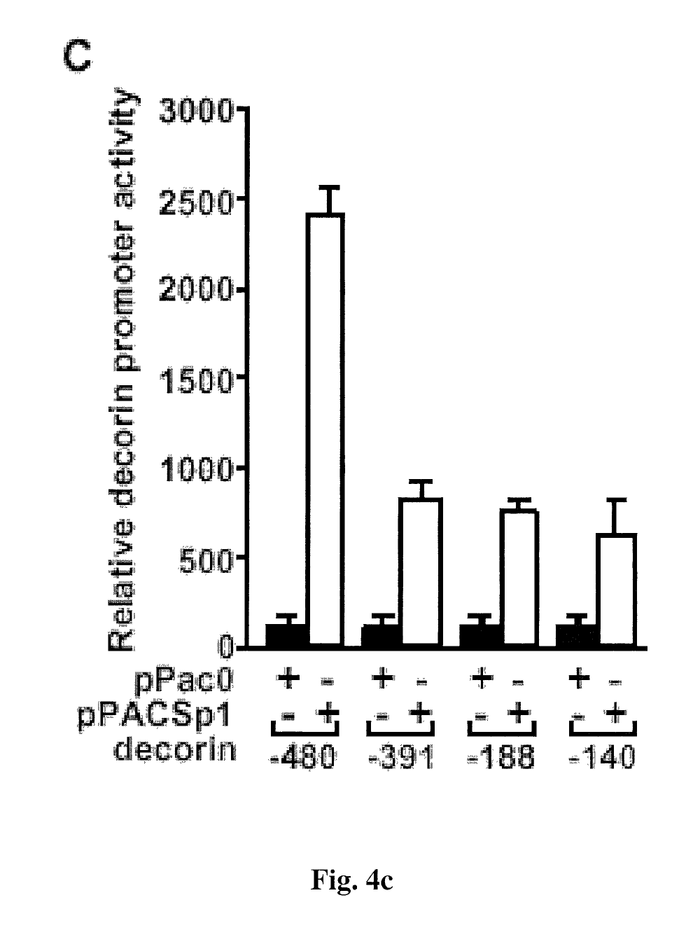

Mammalian cells contain high levels of Sp1 and related transcription factors that contribute to the basal expression of the various genes identified (supra). Therefore, Drosophila melanogaster SL2 cells, the only higher eukaryotic cells known to be devoid of endogenous Sp1 (Courey and Tjian, Cell 55: 887-898, 1988), are used to further characterize the role played by Sp1 cis-elements on the activity of the various promoters studied (supra). In addition to the absence of Sp1 protein, SL2 cells display a high degree of conservation of the transcriptional machinery with mammalian cells, thereby allowing for the functional examination of transfected Sp1. (Hagen et al, EMBO J 13: 3843-3851, 1994). This high degree of conservation of these unique properties of SL2 cells has been previously used to analyze Sp1 functions in the context of various collagen promoters. (Li et al, Gene 164: 229-1234. 1995; Chen et al, Gene 215: 101-110, 1998; Vindevoghel et al, J Biol Chem 272: 10196-10204, 1997). First, SL2 cells are co-transfected with the same 5′ end deletion constructs of the COL1A2 promoter linked to the CAT gene described in FIG. 3, together with an Sp1 expression vector, pPacSp1 or its empty counterpart, pPac0. CAT activity is measured 40 h later. As shown in FIG. 4A, all constructs exhibit identical low basal activity in SL2 cells, a result which contrasts those obtained in human dermal fibroblasts (see FIG. 3) and consistent with the absence of Sp1 in SL2 cells. Overexpression of Sp1 transactivates the constructs with increasing effectiveness as the constructs extended 5′ to include an increasing number of functional Sp1 sites. These results are the reverse image of those presented in FIG. 3, and emphasize the critical role of the Sp1 cis-elements (supra). These data also indicate that yet to be identified upstream elements between nucleotides −376 and −3500 significantly contribute to Sp1-driven COL1A2 expression (supra), in addition to the three characterized Sp1 sites between nucleotide positions −342 and −271. (Inagaki et al, J Biol Chem 269: 14828-14834, 1994; Tamaki et al, J Biol Chem 270 4299-4304, 1995; Chung et al, J Biol Chem 271: 3272-3278, 1996).

Several 5′ end deletion constructs of the COL3A1, decorin and TIMP-1 genes are also tested for their responsiveness to transfected Sp1 in SL2 cells. As shown in FIGS. 4B-D, maximal activation of the promoters is achieved with the largest constructs, likely reflecting their higher content in Sp1 binding sites as compared to the shorter constructs.

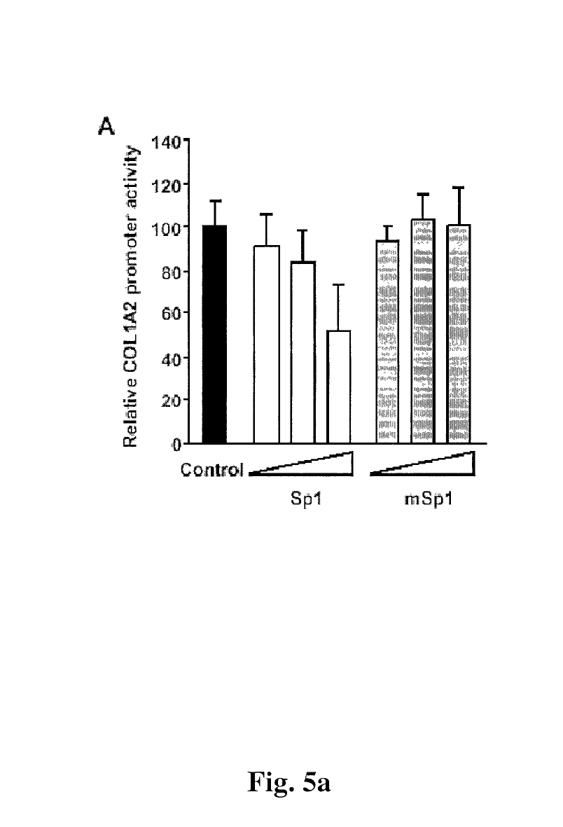

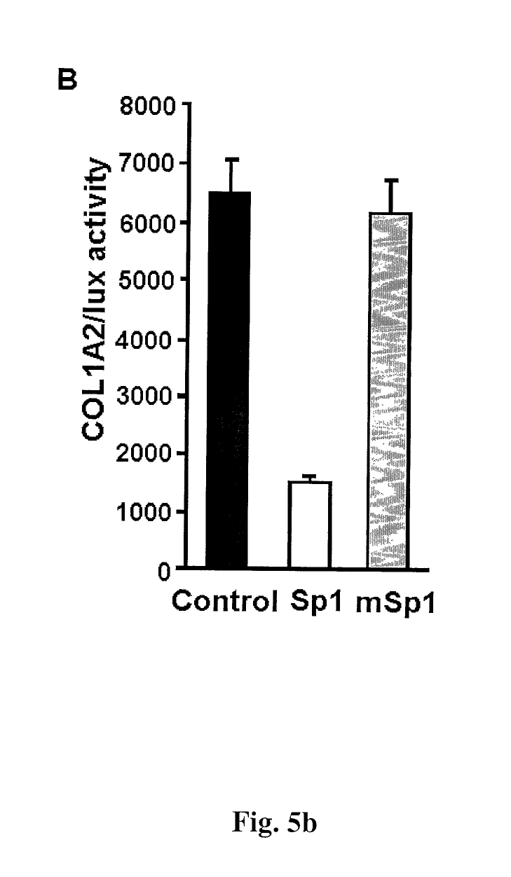

Decoy Sp1 oligonucleotides inhibit COL1A2 promoter activity in vitro and in vivo. Since Sp1 is fundamental for high expression of several ECM genes, short oligonucleotides bearing a consensus Sp1 binding sequence are analyzed for their efficacy as decoys to decrease collagen promoter activity. For this purpose. two approaches are used. First, human dermal fibroblasts are transfected with the −3500COL1A2/CAT construct, and various amounts of either wild-type or mutant Sp1 double-stranded oligonucleotides (SEQ. ID. NO:2 and SEQ. ID. NO: 3. respectively) are added to the culture medium. Promoter activity is determined 24 h later. Results presented in FIG. 5A. indicate that Sp1 oligonucleotide (SEQ. ID. NO: 2), but not mSp1 (SEQ. ID. NO: 3) in which the Sp1 binding site is incapable of binding Sp1, as determined in EMSA experiments, efficiently decreases COL1A2 promoter activity in a dose-dependent manner. Maximal inhibition (55%) is observed with addition of 15 μg/plate of Sp1 oligonucleotide. The COL7A1 promoter is equally inhibited by Sp1 decoy oligonucleotides. No cellular toxicity is observed with either oligonucleotide at any of the concentrations tested.

Next, decoy Sp1 experiments are performed in a transgenic mouse model in which 17 kb of the mouse COL1A2 promoter drives the expression of the luciferase gene. (Bou-Gharios et al, J Cell Biol 134: 1333-1344, 1996). Specifically, 15 μg of either wild-type or mutant Sp1 oligonucleotides (SEQ. ID. NO: 2 and SEQ. ID. NO: 3, respectively) are injected intradermally to the back of 1-month old COL1A2/lux transgenic mice. Luciferase activity is determined 24 hours later in the skin region surrounding the injections. High levels of luciferase activity are found in control skin extracts (FIG. 5B), consistent with previously published observations. (Bou-Gharios et al, J Cell Biol 134: 1333-1344, 1996). The wild-type Sp1 oligonucleotide (SEQ. ID. NO: 2) reduces COL1A2 promoter activity by about 70%, whereas the mutant oligonucleotide (SEQ. ID. NO: 3) has no effect.

These data imply that decoy Sp1 oligonucleotides diminish collagen promoter activity both in vitro and in vivo.

Therapeutic Methods and Compositions

The invention provides methods of treatment and prophylaxis by administration to a subject of an effective amount of a therapeutic agent, i.e., antisense Sp1 or a decoy Sp1 oligonucleotide. In a preferred aspect, the therapeutic agent is substantially purified. The subject is preferably an animal, including but not limited to animals such as cows, pigs, chickens, etc., and is preferably a mammal, and most preferably human.

Various delivery systems are known and can be used to administer the therapeutic agent of the invention, e.g., encapsulation in liposomes, microparticles, microcapsules, receptor-mediated endocytosis (see, e.g., Wu and Wu, , J. Biol. Chem. 262: 4429-4432, 1987), construction of a therapeutic nucleic acid as part of a retroviral or other vector, etc. Methods of introduction include but are not limited to intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers.

The present invention also provides pharmaceutical compositions. Such compositions comprise a therapeutically effective amount of the therapeutic agent, and a pharmaceutically acceptable carrier or excipient. Such a carrier includes but is not limited to saline, buffered saline, dextrose, water, glycerol, ethanol, and combinations thereof. The carrier and composition can be sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutic agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic agent of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Antisense Regulation of ECM Gene Expression

The present invention provides the therapeutic or prophylactic use of nucleic acids that are antisense to a gene or cDNA encoding the Sp1 transcription factor or any portion thereof. Such antisense nucleic acids have utility as therapeutic agents and can be used in the treatment or prevention of fibrotic disorders, including, but not limited to, cirrhosis, radiation induced fibrosis, skin disorders (for example, but not limited to: sclerodermic lesions, hypertrophic scars, keloids), kidney fibrosis, lung fibrosis and myelofibrosis. In addition, the methods of the present invention are used to treat or prevent fibrotic scarring following trauma or surgery.

The antisense nucleic acids of the invention are oligonucleotides that are single-stranded, RNA or DNA or a modification or derivative thereof, which can be directly administered to a cell, or which can be produced intracellularly by transcription of exogenous, introduced sequences. The invention is directed to methods for inhibiting the expression of an Sp1 nucleic acid sequence in a cell by providing the cell with an effective amount of the antisense Sp1 nucleic acid of the invention.

In an alternative embodiment, the Sp1 antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the Sp1 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the Sp1 antisense RNA can be by any promoter known in the art to act in mammalian, preferably human, cells. Such promoters can be inducible or constitutive. Such promoters include but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981. Proc. Natl. Acad. Sci. U.S.A. 78: 1441-1445), the regulatory sequences of the metallothionein gene (Brinster et al., 1982, Nature 296: 3942), etc.

The antisense nucleic acids of the invention comprise a sequence complementary to at least a portion of an RNA transcript of an Sp1 gene, preferably a human Sp1 gene. As an example of an antisense nucleic acid, the fragment spanning +47 to +281 of the human Sp1 gene is used. Other antisense sequences that are complementary to a portion of an Sp1 RNA transcript are also part of the present invention. One skilled in the art can determine which portion of the Sp1 transcript to target for antisense inhibition. However, absolute complementarity, although preferred, is not required. A sequence “complementary to at least a portion of an RNA,” as referred to herein, means a sequence having sufficient complementarity to be able to hybridize with the RNA, forming a stable duplex. The ability to hybridize will depend on both the degree of complementarity and the length of the antisense nucleic acid. Generally, the longer the hybridizing nucleic acid, the more base mismatches with an Sp1 RNA it may contain and still form a stable duplex (or triplex, as the case may be). One skilled in the art can ascertain a tolerable degree of mismatch by use of standard procedures to determine the melting point of the hybridized complex.

Decoy Oligonucleotides

The decoy oligonucleotides of the present invention are oligonucleotides of any sequence that contains at least one Sp1 binding site, including, but not limited to that of SEQ. ID. NO: 2. Also, the Sp1 binding site can be from any genes promoter region. These decoy oligonucleotides have utility as therapeutic agents and are used in the treatment or prevention of fibrotic disorders, including, but not limited to, cirrhosis, skin disorders (for example, but not limited to: sclerodermic lesions, hypertrophic scars, keloids), and myelofibrosis. In addition, the methods of the present invention are used to treat or prevent fibrotic scarring following trauma or surgery.

The double-stranded oligonucleotides of the present invention can be DNA or chimeric mixtures or derivatives or modified versions thereof. The oligonucleotide can be modified at the base moiety, sugar moiety, or phosphate backbone. The oligonucleotide may include other appending groups such as peptides, or agents facilitating transport across the cell membrane (see, e.g., Letsinger et al., Proc. Natl. Acad. Sci. U.S.A. 86: 6553-6556, 1989; Lemaitre et al., Proc. Natl. Acad. Sci. U.S.A. 84: 648-652, 1987; PCT Publication No. WO 88/09810, published Dec. 15, 1988) or blood-brain barrier (see,e.g., PCT Publication No. WO 89/10134, published Apr. 25, 1988), hybridization-triggered cleavage agents (see, e.g., Krol et al., BioTechniques 6: 958-976, 1988) or intercalating agents (see, e.g., Zon, Pharm. Res. 5: 539-549, 1988).

The oligonucleotide may be conjugated to another molecule, e.g., a peptide. hybridization triggered cross-linking agent, transport agent, hybridization-triggered cleavage agent, etc.

In designing the oligonucleotides of the invention, in addition to incorporating the deoxynucleotides, nucleotides, or analogs thereof required to make up the Sp1 recognition sequence(s), “spacer” nucleotides can also be incorporated into the oligonucleotide. Thus, the invention oligonucleotide can include additional nucleotide sequences which are not part of the Sp1 recognition sequence(s). While there is no requirement that spacer nucleotides be incorporated into the oligonucleotide of the present invention, up to 30 nucleotides or more can be present, in addition to the Sp1 recognition sequence(s).

While the oligonucleotides of the invention contain at least one Sp1 recognition sequence, those of skill in the art recognize that the invention compositions can contain multiple Sp1 recognition sequences. Such oligonucleotides can contain multiple repeats of the same Sp1 recognition sequence, or one or more copies of more than one Sp1 recognition sequence. While, in theory, there is no limit as to the number of Sp1 recognition sequences which can be included in a single oligonucleotide of the invention, generally, ten or fewer Sp1 recognition sequences will be included in a single oligonucleotide.

Oligonucleotides of the invention may be synthesized by standard methods known in the art, e.g., by use of an automated DNA synthesizer (such as are commercially available from Biosearch, Applied Biosystems, etc.). A single strand of DNA having the desired sequence of nucleotides and/or nucleotide analogs and the corresponding complementary strand(s) prepared on the DNA synthesizer are then allowed to self-associate.

Oligonucleotides contemplated for use in the practice of the present invention can be prepared from naturally occurring nucleotides or deoxynucleotides (A, C, G, T, or U), as well as nuclease resistant analogs thereof (e.g., phosphorothioates, methylphosphonates, phosphoramidates, and the like). As examples, phosphorothioate oligos may be synthesized by the method of Stein et al. (Nucl. Acids Res. 16: 3209, 1988), methylphosphonate oligos can be prepared by use of controlled pore glass polymer supports (Sarin et al., Proc. Natl. Acad. Sci. U.S.A. 85: 7448-7451, 1988). etc.

Discussion

The transcription factor Sp1 is critical for basal expression of several ECM genes. Using several complementary approaches, the present invention demonstrates that targeting Sp1 represents a powerful therapeutic approach to reduce ECM accumulation in fibrotic conditions. Two independent experimental approaches (supra) targeting Sp1, reveal that interference with either the expression of Sp1 (via antisense technology) or the DNA binding activity of Sp1 (via decoy oligonucleotides) results in transcriptional repression of several ECM genes.

NIH-3T3 clones stably transfected with pRSV/ASSp1 are selected and indicate that a reduced Sp1 expression is compatible with cell survival and growth and leads to simultaneous reduction in the mRNA steady-state levels of numerous cellular genes, including, but not limited to, those encoding fibrillar collagens, proteoglycans and other ECM proteins. This inhibition results from a reduction in the transcriptional activity, as measured using transient cell transfections with several ECM promoter/reporter gene constructs. Further, using decoy Sp1 oligonucleotides the present invention demonstrates that interfering with Sp1 binding to its target sequences, such as within the COL1A2 promoter, efficiently blocks gene expression both in vitro and in vivo. These data are obtained using either classic transient cell transfections of dermal fibroblasts in cultures or a transgenic mouse model that allows in vivo monitoring of COL1A2 promoter activity. (Bou-Gharios et al, J Cell Biol 134: 1333-1344, 1996). At the sites of injection in the skin of these transgenic animals the Sp1 oligonucleotide decoy effectively competes with the cellular transcriptional machinary to efficiently decrease COL1A2 promoter activity Simultaneous monitoring of the expression of hundreds of genes (863) using cDNA microarrays allows for the identification of numerous novel Sp1 targets (Tables I and II). Interestingly, expression of the various cyclin genes is selectively inhibited; a phenomenon which explains why pRSV/ASSp1 clones have proliferation rates indiscernible from that of the control clones.

A perfect correlation between decreased mRNA steady-state levels and reduced promoter activity for several ECM genes is observed (supra). Further, several of the genes that are identified using cDNA arrays have been previously shown to be under the transcriptional control of Sp1, including, but not limited to, TGF-β1 (Geiser et al, Gene 129: 223-228, 1993), cyclinD1 (Kitazawa et al, J Biol Chem 274: 28787-93, 1999), or the cyclin-dependent kinase inhibitors p21/Waf-1. (Moustakas and Kardassis, Proc Natl Acad Sci USA 95: 7633-6738, 1998) and p27 (Zhang and Lin, Biochim Biophys Acta 1353: 307-317, 1997).

Targeting Sp1 will also allow for the modulation of transforming growth factor-β (TGF-β-driven gene expression). TGF-β is the prototypic fibrogenic cytokine, enhancing ECM gene expression and downregulating that of matrix-degrading enzymes. Increased expression of TGF-3 is often associated with fibrotic states and abnormal accumulation of ECM. (review in Branton and Kopp, Microbes Infect 1: 1349-1365, 1999). Initiation of cellular signaling of the TGF-β family of growth factors occurs through specific serine/threonine kinase transmembrane receptors which, upon activation by TGF-β, phosphorylate cytoplasmic mediators of the Smad family. The latter then translocate into the nucleus where they act as transcription factors. (reviewed in Piek et al, FASEB J 13: 2105-2124, 1999; Massagné and Wotton, EMBO J 19: 1745-1754, 2000). It has been shown that Smad-mediated activation of the p15 and p21 gene by TGF-β requires direct Smad-Sp1 interactions. (Pardali et al, J Biol Chem 275: 29244-29256, 2000; Feng et al, EMBO J 19: 5178-5193, 2000). It has also been proposed that Sp1 is necessary for TGF-β-driven activation of the human COL1A2 promoter (Greenwell et al, J Biol Chem. 272: 19738-19745, 1997), although 5′ deletion studies suggest that the effector essential for basal activity of the COL1A2 promoter is TGF-β, independent of Sp1. (Chung et al, J Biol Chem 271: 3272-3278, 1996). Alteration of the TGF-β signal by Sp1 targeting may also be obtained by direct repression of the TGF-β1 gene, whose activity is influenced by Sp1 cis-elements. (Geiser et al, Gene 129: 223-228, 1993). This result is supported by the strong repression of TGF-β1 expression in the cDNA microarrays (supra and Table I). Whether directly interfering with the Smad signaling cascade or with TGF-β expression, Sp1 targeting is useful to block the fibrogenic properties of TGF-β.

Pharmacologic targeting of Sp1 has been shown to inhibit collagen gene expression. For example, mithramycin, a DNA-intercalating tumor antibiotic with high affinity for GC-rich sequences, specifically inhibits COL1A1 gene expression in cultured fibroblasts by preventing Sp1, but not NF-1, binding to its cognate cis-elements. (Nehls et al, J Clin Invest 92: 2916-2921, 1993). The use of this drug for the treatment of fibrosis is impossible, due to its cell and tissue toxicity, as well as its mutagenicity. Mithramycin has been shown to induce mutants in the phosphoribosyl-transferase locus and to produce DNA strand breaks in vitro. (Singh and Gupta, Cancer Res 45: 2813-2820, 1985). Mithramycin is also known to induce hepatic and renal toxicity associated with severe platelet dysfunction in patients undergoing tumor chemotherapy or treated for tumor-induced hypercalcemia. (reviewed in Weiss and Poster, Cancer Treat Rev 9: 37-56, 1982; Zojer et al, Drug Saf 21: 389-406, 1999).

Sp1 is the target of choice for the establishment of novel therapeutic treatments of fibrotic states. The invention disclosed herein uses antisense technology to repress the expression of Sp1, thereby inhibiting the transcriptional activation of ECM genes. The present invention further relates to decoy oligonucleotides, wherein the decoy oligonucleotides interfere with Sp1 binding to its target DNA (including, but not limited to, type I collagen, type III collagen, type V collagen, type VII collagen, various proteoglycans, TGF-β, etc.) thereby interfering with the transcriptional activation of that gene. The present invention also relates to a method of treating a mammal, wherein treatment with an Sp1 decoy oligonucleotide or an antisense Sp1 will effectively reduce ECM gene expression. The inhibition of accumulation of excess collagen and other extracellular matrix components by the methods of the present invention allows for a novel therapeutic approach to treating fibrillar conditions or disorders. The methods of the present invention fulfill a long felt need in the treatment and prevention of fibrotic conditions, since presently there are few effective therapies for collagen disorders.

What is claimed is:

1. A method of treating a fibrotic condition wherein the transcription of an ECM gene is inhibited in a mammal, comprising
a) administering to said mammal a therapeutically effective amount of antisense Sp1;
b) binding of said antisense Sp1 to an Sp1 transcript;
c) reducing Sp1 expression;
d) inhibiting transcription of said ECM gene; and
e) reducing accumulation of a corresponding ECM protein.

2. The method of claim 1, wherein said antisense Sp1 has the nucleic acid sequence of SEQ. ID. NO: 1.

3. A recombinant expression vector, comprising a XhoI/HindIII DNA fragment of an Sp1 gene (SEQ. ID. NO: 1) in an antisense orientation cloned upstream of a Rous Sarcoma Virus (RSV) promoter.

4. A method of treating a fibrotic condition wherein the gene expression of an ECM gene is inhibited in a mammal, comprising
a) administering to said mammal a therapeutically effective amount of a decoy Sp1 oligonucleotide;
b) binding of an Sp1 transcription factor to said decoy Sp1 oligonucleotide;
c) interfering with Sp1 binding to its target sequences;
d) decreasing promoter activity of said ECM gene;
e) inhibiting gene expression of said ECM gene; and
f) reducing accumulation of a corresponding ECM protein.

5. The method of claim 4, wherein said decoy Sp1 oligonucleotide has the nucleic acid sequence of SEQ. ID. NO: 2.

6. A method of treating a fibrotic condition wherein the gene expression of TGF-β is inhibited in a mammal, comprising
a) administering to said mammal a therapeutically effective amount of a decoy Sp1 oligonucleotide;
b) binding of an Sp1 transcription factor to said decoy Sp1 oligonucleotide;
c) interfering with Sp1 binding to its target sequences;
d) decreasing promoter activity of said TGF-β gene;
e) inhibiting gene expression of said TGF-β gene; and
f) blocking fibrogenic properties of TGF-β.

7. The method of claim 6, wherein said decoy Sp1 oligonucleotide has the nucleic acid sequence of SEQ. ID. NO: 2.
